(19) United States
(12) Patent Application Publication
(10) Pub. No.: US 2019/0137516 A1
(43) Pub. Date: May 9, 2019

(54) EVALUATING METHOD FOR FUTURE RISK OF DEVELOPING ALZHEIMER'S DISEASE

(71) Applicant: AJINOMOTO CO., INC., Tokyo (JP)

(72) Inventors: Takeshi Ikeuchi, Niigata (JP); Yuki Yano, Kanagawa (JP); Naoko Arashida, Kanagawa (JP); Rumi Nishimoto, Kanagawa (JP); Kazutaka Shimbo, Kanagawa (JP); Nobuhiro Kawai, Kanagawa (JP)

(73) Assignee: AJINOMOTO CO., INC., Tokyo (JP)

(21) Appl. No.: 16/239,834

(22) Filed: Jan. 4, 2019

Related U.S. Application Data
(63) Continuation of application No. PCT/JP2017/025050, filed on Jul. 7, 2017.

(30) Foreign Application Priority Data
Jul. 8, 2016 (JP) 2016-136339

(51) Int. Cl. G01N 33/68; G16H 10/40; G16H 50/30

(52) U.S. Cl. CPC G01N 33/6896; G16H 50/30; G16H 10/40

(57) ABSTRACT

An evaluating method includes an evaluating step of evaluating future risk of developing Alzheimer's disease for a subject to be evaluated having mild cognitive impairment using a concentration value of at least one of α-ABA, Ala, Arg, Asn, Cit, Gln, Glu, Gly, His, Ile, Leu, Lys, Met, Orn, Phe, Pro, Ser, Thr, Trp, Tyr, Val, Cysteine, Taurine, bABA, Ethylglycine, Hypotaurine, 3-Me-His, 5-HydroxyTrp, aAiBA, and N8-Acetylspermidine in blood of the subject.

### CROSS-REFERENCE TO RELATED APPLICATION

This application is based upon and claims the benefit of priority from PCT Application PCT/JP2017/025050, filed Jul. 7, 2017, which claims priority from Japanese Patent Application No. 2016-136339, filed Jul. 8, 2016, the entire contents of which are incorporated herein by reference.

### BACKGROUND OF THE INVENTION

### 1. Field of the Invention

The present invention relates to an evaluating method, an evaluating apparatus, an evaluating program product, an evaluating system, and a terminal apparatus for future risk of developing Alzheimer's disease (hereinafter, may be referred to as AD).

### 2. Description of the Related Art

Dementia is a condition where the normally developed intellectual function of a patient has been entirely and continuously decreased by acquired brain lesions and the daily living of the patient is affected, and is a disease defined as “a syndrome generally caused by chronic or progressive brain disease, comprising a plurality of disabled higher cerebral functions of memory, thought, orientation, comprehension, calculation, learning, language, and decision” (“International Classification of Diseases, 10th revision, World Health Organization”). It is said that 60% of primary causes of dementia is Alzheimer's disease that is most common.

The typical neuropathological features of AD are neuritic plaques and neurofibrillary tangles. It has been found that the neuritic plaques are caused by deposition of the protein called amyloid β (Aβ)and the neurofibrillary tangles are caused by excessively phosphorylated tau protein. A recent large-scale observation study revealed that these pathological features appear before AD develops (“Jack, et al., Lancet Neurol (2010) 9(1): 119-28.; Hypothetical model of dynamic biomarkers of the Alzheimer's pathological cascade.”). In the recent years, some diagnostic imaging techniques, such as the positron emission tomography (PET), the single photon emission computed tomography (SPECT), and the magnetic resonance imaging (MRI), have been offered as methods for quantifying the accumulation of Aβ and phosphorylated tau protein in brain tissue and quantifying the atrophy of brain tissue. However, independent use of any one of the diagnostic imaging techniques mentioned above is not recommended as a definitive diagnosis method. At present, AD is diagnosed based on total evaluation including a neuropsychological test and clinical symptom observation. Besides, a technique for diagnosing AD using the concentrations of Aβ and phosphorylated tau protein in cerebrospinal fluid (CSF) as indices has been offered (“Knopman, et al., Neurology. (2001) 8; 56(9): 1143-53.; Practice parameter: diagnosis of dementia (an evidence-based review). Report of the Quality Standards Subcommittee of the American Academy of Neurology.”).

The acetylcholinesterase inhibitor and the N-methyl-D-aspartic acid (NMDA) receptor inhibitor are used as a therapeutic agent against AD, but those medicines can merely exert the effect of delaying progression of the disease for a certain period, and a disease modifying therapy necessary for the radical cure has not been established yet. In addition, antibody drugs have been developed based on neuropathological findings in the accumulation of Aβ and phosphorylated tau protein, but candidate medicines exerting remarkable effects have not been found yet. Thus, clinical trials for therapeutic agents against AD and preventive agents for AD intervening in a previous stage before developing AD are increasing in recent years.

Thus, the necessities of early diagnostic before developing AD and prevention of development of AD through the early intervention have increased in recent years. In the present description, mild cognitive impairment (hereinafter, may be referred to as MCI) means a condition considered as a previous stage or a borderline case of various types of dementia that shows severer problems in cognitive function than those of a person of the same age or a normally aging person, but does not affect daily living, being not considered as dementia. At present, the major two diagnostic criteria have been offered and widely accepted (“Petersen, et al., Arch Neurol (1999) 56(6):760.; Mild cognitive impairment: clinical characterization and outcome.” and “Winblad, et al., J. Intern. Med. (2004) 256(3): 240-6.; Mild cognitive impairment--beyond controversies, towards a consensus: report of the International Working Group on Mild Cognitive Impairment.”). It has been revealed that a number of patients diagnosed as MCI develop AD in several years at high probabilities. However, all patients having MCI do not necessarily develop AD. Some patients may develop other types of dementia, such as vascular dementia, Lewy body dementia, and frontotemporal dementia, which are different from AD. Furthermore, methods of treatment differ for the types of dementia. Accordingly, in the stage of developing MCI, it is difficult to determine a proper treatment strategy for the condition of a patient. Therefore, it has been desired that, by providing disease prediction techniques for determining whether symptoms progress from MCI to AD in future for a group with MCI showing cognitive decline caused by various background diseases, the techniques be useful for selecting proper methods for intervention and treatment in an early stage. In addition, it has been desired that, by providing such disease prediction techniques, the techniques be useful for selecting proper subjects for clinical trials for therapeutic drugs and preventive drugs for AD targeting MCI.

In recent years, some studies on the application of diagnostic imaging methods, for example, Amyloid PET imaging and Tau PET imaging used as techniques for diagnosing AD, to an index for predicting the development of AD have been made. It is, however, practically difficult to make a patient without subjective symptom in the preclinical stage have such highly invasive examinations. Thus, it has been desired to provide a simple and inexpensive screening test technique.

Incidentally, some methods of measuring the blood concentration of amino acids and amino acid related metabolites to determine incidence risk based on the features in a particular disease have been known in the diagnosis of diseases such as cancer, metabolic syndrome, and liver disease (JP-A-2014-025946, JP-A-2016-029398, and JP-A-2013-040923). A technique for diagnosing AD based on the blood concentrations of particular amino acids as indices has been developed (JP-A-2011-242217). As a technique for determining MCI based on a blood test, a technique for measuring the blood concentration of peptide fragments for use as indices has been developed (JP-A-2016-028244).

However, there is a problem in that simple and inexpensive techniques of determining the future risk of developing AD from MCI, using the blood concentrations of amino acids and amino acid related metabolites contained in blood obtained by a blood test as indices, have not been developed or put into practice yet.

### SUMMARY OF THE INVENTION

It is an object of the present invention to at least partially solve the problems in the conventional technology.

The present invention has been made in view of the above descriptions, and an object of the present invention is to provide an evaluating method, an evaluating apparatus, an evaluating program product, an evaluating system, and a terminal apparatus, which can provide reliable information that may be helpful in knowing the future risk of developing AD from MCI (risk of progression from MCI to AD).

To solve the problem and achieve the object described above, an evaluating method according to one aspect of the present invention includes an evaluating step of evaluating future risk of developing Alzheimer's disease for a subject to be evaluated having mild cognitive impairment using a concentration value of at least one of 23 kinds of amino acids (α-ABA, Ala, Arg, Asn, Cit, Gln, Glu, Gly, His, Ile, Leu, Lys, Met, Orn, Phe, Pro, Ser, Thr, Trp, Tyr, Val, Cysteine, and Taurine) and 7 kinds of amino acid related metabolites (bABA[3-Aminobutanoic acid], Ethylglycine, Hypotaurine, 3-Me-His[N(tau)-Methyl-L-histidine], 5-HydroxyTrp[5-Hydroxytryptophan], aAiBA[2-Aminoisobutyric acid], N8-Acetylspermidine) in blood of the subject.

In the present description, various amino acids are mainly written in abbreviations, the formal names of these are as follows.

| (Abbreviation) | (Formal name) |
|---|---|
| α-ABA | α-Aminobutyric acid |
| Ala | Alanine |
| Arg | Arginine |
| Asn | Asparagine |
| Cit | Citrulline |
| Gln | Glutamine |
| Glu | Glutamic acid |
| Gly | Glycine |
| His | Histidine |
| Ile | Isoleucine |
| Leu | Leucine |
| Lys | Lysine |
| Met | Methionine |
| Orn | Ornithine |
| Phe | Phenylalanine |
| Pro | Proline |
| Ser | Serine |
| Thr | Threonine |
| Trp | Tryptophan |
| Tyr | Tyrosine |
| Val | Valine |

An evaluating apparatus according to one aspect of the present invention is an evaluating apparatus including a control unit. The control unit includes an evaluating unit that evaluates future risk of developing Alzheimer's disease for a subject to be evaluated having mild cognitive impairment using a concentration value of at least one of the 23 kinds of amino acids and the 7 kinds of amino acid related metabolites in blood of the subject.

An evaluating method according to one aspect of the present invention is an evaluating method executed by an information processing apparatus including a control unit. The evaluating method includes an evaluating step of evaluating future risk of developing Alzheimer's disease for a subject to be evaluated having mild cognitive impairment using a concentration value of at least one of the 23 kinds of amino acids and the 7 kinds of amino acid related metabolites in blood of the subject. The evaluating step is executed by the control unit.

An evaluating program product according to one aspect of the present invention is an evaluating program product having a non-transitory tangible computer readable medium including a programmed instruction for making an information processing apparatus including a control unit execute an evaluating method. The evaluating method includes an evaluating step of evaluating future risk of developing Alzheimer's disease for a subject to be evaluated having mild cognitive impairment using a concentration value of at least one of the 23 kinds of amino acids and the 7 kinds of amino acid related metabolites in blood of the subject. The evaluating step is executed by the control unit.

A recording medium according to one aspect of the present invention is a non-transitory tangible computer-readable recording medium including a programmed instruction for making an information processing apparatus execute the evaluating method.

An evaluating system according to one aspect of the present invention is an evaluating system including an evaluating apparatus including a control unit and a terminal apparatus including a control unit that are connected to each other communicatively via a network. The control unit of the terminal apparatus includes (i) a concentration data-sending unit that transmits concentration data on a concentration value of at least one of the 23 kinds of amino acids and the 7 kinds of amino acid related metabolites in blood of a subject to be evaluated having mild cognitive impairment, to the evaluating apparatus and (ii) a result-receiving unit that receives an evaluation result on future risk of developing Alzheimer's disease for the subject, transmitted from the evaluating apparatus. The control unit of the evaluating apparatus includes (i) a concentration data-receiving unit that receives the concentration data transmitted from the terminal apparatus, (ii) an evaluating unit that evaluates the future risk of developing Alzheimer's disease for the subject using the concentration value of the at least one of the amino acids and the amino acid related metabolites included in the concentration data received by the concentration data-receiving unit, and (iii) a result-sending unit that transmits the evaluation result obtained by the evaluating unit to the terminal apparatus.

A terminal apparatus according to one aspect of the present invention is a terminal apparatus including a control unit. The control unit includes a result-obtaining unit that obtains an evaluation result on future risk of developing Alzheimer's disease for a subject to be evaluated having mild cognitive impairment. The evaluation result is the result of evaluating the future risk of developing Alzheimer's disease for the subject using a concentration value of at least one of the 23 kinds of amino acids and the 7 kinds of amino acid related metabolites in blood of the subject.

The terminal apparatus according to another aspect of the present invention is the terminal apparatus, wherein the apparatus is communicatively connected via a network to an evaluating apparatus that evaluates the future risk of developing Alzheimer's disease for the subject. The control unit further includes a concentration data-sending unit that transmits concentration data on the concentration value of the at least one of the amino acids and the amino acid related metabolites to the evaluating apparatus. The result-obtaining unit receives the evaluation result transmitted from the evaluating apparatus.

An evaluating apparatus according to one aspect of the present invention is an evaluating apparatus including a control unit, being connected to a terminal apparatus communicatively via a network. The control unit includes (i) a concentration data-receiving unit that receives concentration data on a concentration value of at least one of the 23 kinds of amino acids and the 7 kinds of amino acid related metabolites in blood of a subject to be evaluated having mild cognitive impairment, transmitted from the terminal apparatus, (ii) an evaluating unit that evaluates future risk of developing Alzheimer's disease for the subject using the concentration value of the at least one of the amino acids and the amino acid related metabolites included in the concentration data received by the concentration data-receiving unit, and (iii) a result-sending unit that transmits an evaluation result obtained by the evaluating unit to the terminal apparatus.

According to the present invention, reliable information that may be helpful in knowing the future risk of developing AD from MCI (the risk of progression from MCI to AD) can be provided.

The above and other objects, features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a principle configurational diagram showing a basic principle of a first embodiment;

FIG. 2 is a principle configurational diagram showing a basic principle of a second embodiment;

FIG. 3 is a diagram showing an example of an entire configuration of a present system;

FIG. 4 is a block diagram showing an example of a configuration of an evaluating apparatus 100 in the present system;

FIG. 5 is a chart showing an example of information stored in a concentration data file 106a;

FIG. 6 is a chart showing an example of information stored in an evaluation result file 106b;

FIG. 7 is a block diagram showing a configuration of an evaluating part 102b; and

FIG. 8 is a block diagram showing an example of a configuration of a client apparatus 200 in the present system.

### DETAILED DESCRIPTION OF THE PREFERRED EMBODIMENTS

Hereinafter, an embodiment (first embodiment) of the evaluating method according to the present invention and an embodiment (second embodiment) of the evaluating apparatus, the evaluating method, the evaluating program product, the recording medium, the evaluating system, and the terminal apparatus according to the present invention are described in detail with reference to the drawings. The present invention is not limited to these embodiments.

### First Embodiment

1-1. Outline of First Embodiment

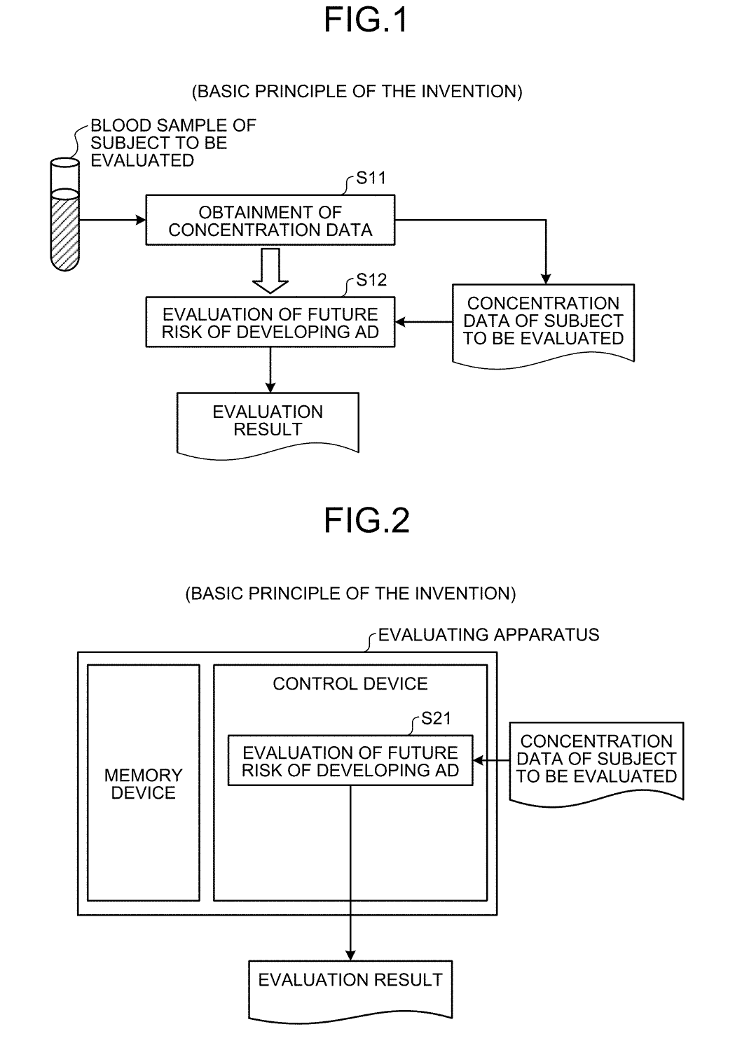

Here, an outline of the first embodiment will be described with reference to FIG. 1. FIG. 1 is a principle configurational diagram showing a basic principle of the first embodiment.

First, concentration data on a concentration value of at least one of the 23 kinds of amino acids and the 7 kinds of amino acid related metabolites (a substance or substances arbitrarily selected from the 23 kinds of amino acids and the 7 kinds of amino acid related metabolites) contained in the blood (including, for example, plasma or serum) extracted from a subject to be evaluated (for example, an individual such as animal or human) having MCI is obtained (Step S11). In the present description, the subject to be evaluated having MCI is, for example, a subject diagnosed as MCI according to an existing diagnostic criterion for MCI (for example, “Petersen, et al., Arch Neurol (1999) 56(6): 760.; Mild cognitive impairment: clinical characterization and outcome.”).

At step S11, for example, the concentration data measured by a company or other organization that measures concentrations may be obtained. In addition, for example, the following measuring method of (A), (B), or (C) may be used to measure the concentration from the blood extracted from the subject to obtain the concentration data. In the present description, the unit of the concentration may be molar concentration, weight concentration, enzyme activity, or one obtained by addition, subtraction, multiplication, and division of any constant with these concentrations. When the measuring method of (A) is used, the peak area or the peak height of each substance in a chromatogram obtained by a mass spectrometer may be used as a substitute for a concentration.

(A) Plasma is separated from blood by centrifuging the collected blood sample. All plasma samples are frozen and stored at −80° C. until the concentration is measured. At the time of measuring the concentration, acetonitrile is added to deproteinize the plasma samples, pre-column derivatization is then performed using a labeling reagent (3-aminopyridyl-N-hydroxysuccinimidyl carbamate), and the concentration is analyzed by liquid chromatograph mass spectrometer (LC/MS) (see International Publication WO 2003/069328 and International Publication WO 2005/116629).

(B) Plasma is separated from blood by centrifuging the collected blood sample. All plasma samples are frozen and stored at −80° C. until the concentration is measured. At the time of measuring the concentration, sulfosalicylic acid is added to deproteinize the plasma samples, and the concentration is analyzed by an amino acid analyzer based on post-column derivatization using a ninhydrin reagent.

(C) Blood cell separation is performed on the collected blood sample by using a membrane, MEMS (Micro Electro Mechanical Systems) technology, or the principle of centrifugation, whereby plasma or serum is separated from the blood. A plasma or serum sample the concentration of which is not measured immediately after obtaining the plasma or the serum is frozen and stored at −80° C. until the concentration is measured. At the time of measuring the concentration, a molecule that reacts with or binds to a target amino acid or amino acid related metabolite, such as an enzyme or an aptamer, and the like are used to perform quantitative analysis and the like on an increasing or decreasing substance or a spectroscopic value by substrate recognition, whereby the concentration is analyzed.

Future risk of developing AD for the subject is evaluated using the concentration value of at least one of the 23 kinds of amino acids and the 7 kinds of amino acid related metabolites included in the concentration data obtained at step S11 (step S12). Before step S12 is executed, data such as defective and outliers may be removed from the concentration data obtained at step S11. In the present description, the evaluation of the future risk of developing AD for the subject to be evaluated is, for example, to predict or examine the future risk of onset of AD in the subject. In the present description, future is, for example, a point of time after a certain time from blood collection (for example, a period known in the medical field as “the average period of developing Alzheimer's disease from MCI”, or a period of several years, for example, three, four, or five years).

According to the first embodiment described above, the concentration data of the subject having MCI is obtained at step S11, and at step S12, the future risk of developing AD for the subject is evaluated using the concentration value of at least one of the 23 kinds of amino acids and the 7 kinds of amino acid related metabolites included in the concentration data of the subject obtained at step S11. In this way, reliable information that may be helpful in knowing the future risk of developing AD can be provided for purpose of avoiding the development of AD, in a previous stage, for example, the MCI stage. The evaluating method according to the present embodiment is valuable as a simple and inexpensive method of testing the risk of developing AD suitable for mass screening.

It may be decided that the concentration value of at least one of the 23 kinds of amino acids and the 7 kinds of amino acid related metabolites reflects the future risk of developing AD for the subject. The concentration value may be converted, for example, by the methods listed below, and it may be decided that the converted value reflects the future risk of developing AD for the subject. In other words, the concentration value or the converted value may be treated per se as an evaluation result on the future risk of developing AD for the subject.

The concentration value may be converted such that a possible range of the concentration value falls within a predetermined range (for example, the range from 0.0 to 1.0, the range from 0.0 to 10.0, the range from 0.0 to 100.0, or the range from −10.0 to 10.0), for example, by addition, subtraction, multiplication, and division of any given value with the concentration value, by conversion of the concentration value by a predetermined conversion method (for example, exponential transformation, logarithm transformation, angular transformation, square root transformation, probit transformation, reciprocal transformation, Box-Cox transformation, or power transformation), or by performing a combination of these computations on the concentration value. For example, a value of an exponential function with the concentration value as an exponent and Napier constant as the base may be further calculated (specifically, a value of p/(1-p) where a natural logarithm 1n(p/(1-p)) is equal to the concentration value when the probability p that the future risk of developing AD has a predetermined state (for example, a state of high risk) is defined), and a value (specifically, a value of the probability p) may be further calculated by dividing the calculated value of the exponential function by the sum of 1 and the value of the exponential function.

The concentration value may be converted such that the converted value is a particular value when a particular condition is met. For example, the concentration value may be converted such that the converted value is 5.0 when the sensitivity is 95% and the converted value is 8.0 when the sensitivity is 80%.

For each amino acid and each amino acid related metabolite, after normally distributing the concentration distribution, the concentration value may be standardized with a mean of 50 and a standard deviation of 10.

These conversions may be performed by gender or age.

The future risk of developing AD for the subject may be evaluated using the concentration value converted, for example, by the conversion method described above.

Positional information about a position of a predetermined mark on a predetermined scale visually presented on a display device such as a monitor or a physical medium such as paper may be generated using the concentration value of at least one of the 23 kinds of amino acids and the 7 kinds of amino acid related metabolites or, if the concentration value is converted, the converted value, and it may be decided that the generated positional information reflects the future risk of developing AD for the subject. The predetermined scale is for evaluating the future risk of developing AD and is, for example, a graduated scale at least marked with graduations corresponding to the upper limit value and the lower limit value in “a possible range of the concentration value or the converted value”, or “part of the range”. The predetermined mark corresponds to the concentration value or the converted value and is, for example, a circle sign or a star sign.

If the concentration value of at least one of the 23 kinds of amino acids and the 7 kinds of amino acid related metabolites is lower than a predetermined value (e.g., mean±1SD, 2SD, 3SD, N quantile, N percentile, or a cutoff value the clinical significance of which is recognized) or is equal to or lower than the predetermined value, or the concentration value is equal to or higher than the predetermined value or is higher than the predetermined value, the future risk of developing AD for the subject may be evaluated. In this case, instead of the concentration value itself, a standard score (a value obtained by normally distributing the concentration distribution by gender and then standardizing the concentration value with a mean of 50 and a standard deviation of 10 for each amino acid and each amino acid related metabolite) may be used. For example, if the concentration standard score is lower than the mean −2SD (when the concentration standard score<30) or if the concentration standard score is higher than the mean +2SD (when the concentration standard score>70), the future risk of developing AD for the subject may be evaluated.

The degree of the risk (possibility) of future AD of the subject may be qualitatively evaluated. Specifically, the subject may be classified into any one of a plurality of categories defined at least considering the degree of the future risk of developing AD, using the concentration value of at least one of the 23 kinds of amino acids and the 7 kinds of amino acid related metabolites, and one or more preset thresholds. The categories may include (i) a category to which a subject whose future risk of developing AD is high belongs, (ii) a category to which a subject whose future risk of developing AD is low belongs, and (iii) a category to which a subject whose future risk of developing AD is intermediate belongs. The categories may include (i) the category to which a subject whose future risk of developing AD is high belongs, and (ii) the category to which a subject whose future risk of developing AD is low belongs. The concentration value may be converted by the predetermined method, and the subject may be classified into any one of the categories using the converted value.

When the future risk of developing AD is evaluated, a value related to other biological information listed below may further be used in addition to the concentration value of at least one of the 23 kinds of amino acids and the 7 kinds of amino acid related metabolites.
1. Concentration values of metabolites in blood other than amino acids (e.g., amino acid metabolites, carbohydrates, and lipids), proteins, peptides, minerals, vitamins, organic acids, hormones, and the like.
2. Blood test values such as albumin, total protein, triglyceride (neutral fat), HbAlc, glycoalbumin, insulin resistance index, total cholesterol, LDL cholesterol, HDL cholesterol, amylase, total bilirubin, creatinine, estimated glomerular filtration rate (eGFR), uric acid, GOT (AST), GPT (ALT), GGTP (γ-GTP), glucose (glucose level), CRP (C-reactive protein), erythrocyte, hemoglobin, hematocrit, MCV, MCH, MCHC, leucocyte, and the number of thrombocytes.
3. Values obtained from image information such as ultrasonic echo, X ray, CT (Computed Tomography), MRI (Magnetic Resonance Imaging), and endoscope image.
4. Values of biological indices such as age, height, weight, BMI, abdominal girth, systolic blood pressure, diastolic blood pressure, gender, smoking information, dietary information, drinking information, exercise information, stress information, sleeping information, family medical history information, and disease history information (for example, diabetes).
5. Values obtained from gene information, for example, number of risk genes for Alzheimer's disease (for example, APOEε4 allele).

### Second Embodiment

2-1. Outline of the Second Embodiment

Here, outlines of the second embodiment will be described in detail with reference to FIG. 2. FIG. 2 is a principle configurational diagram showing a basic principle of the second embodiment. In the description of the present second embodiment, description duplicating that of the first embodiment is sometimes omitted.

A control device evaluates the future risk of developing AD for the subject having MCI using the concentration value of at least one of the 23 kinds of amino acids and the 7 kinds of amino acid related metabolites included in the previously obtained concentration data of the subject (for example, an individual such as animal or human) on the concentration value of at least one of the 23 kinds of amino acids and the 7 kinds of amino acid related metabolites in blood (step S21). In this way, reliable information that may be helpful in knowing the future risk of developing AD can be provided for purpose of avoiding the development of AD, in a previous stage, for example, the MCI stage.

2-2. System Configuration

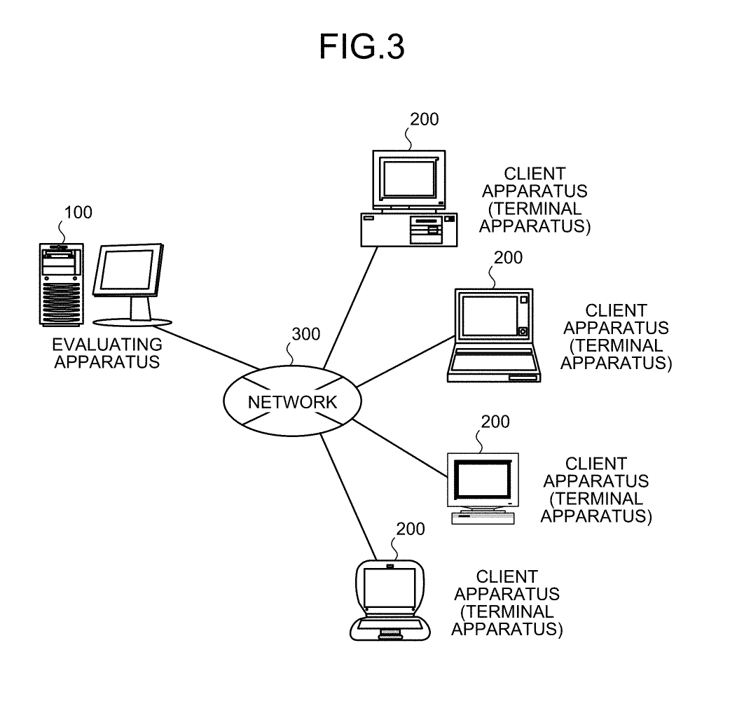

Hereinafter, the configuration of the evaluating system according to the second embodiment (hereinafter referred to sometimes as the present system) will be described with reference to FIGS. 3 to 8. This system is merely one example, and the present invention is not limited thereto.

First, an entire configuration of the present system will be described with reference to FIG. 3. FIG. 3 is a diagram showing an example of the entire configuration of the present system. As shown in FIG. 3, the present system is constituted in which the evaluating apparatus 100 that evaluates the future risk of developing AD for the individual as the subject and the client apparatus 200 (corresponding to the terminal apparatus of the present invention) that provides the concentration data of the individual are communicatively connected to each other via a network 300.

The network 300 has a function to connect the evaluating apparatus 100 and the client apparatuses 200 mutually, communicatively to one another, and is for example the Internet, an intranet, or LAN (Local Area Network (including both wired and wireless)). The network 300 may be VAN (Value Added Network), a personal computer communication network, a public telephone network (including both analog and digital), a leased line network (including both analog and digital), CATV (Community Antenna Television) network, a portable switched network or a portable packet-switched network (including IMT2000 (International Mobile Telecommunication 2000) system, GSM (registered trademark) (Global System for Mobile Communications) system, or PDC (Personal Digital Cellular)/PDC-P system), a wireless calling network, a local wireless network such as Bluetooth (registered trademark), PHS network, a satellite communication network (including CS (Communication Satellite), BS (Broadcasting Satellite), ISDB (Integrated Services Digital Broadcasting), and the like), or the like.

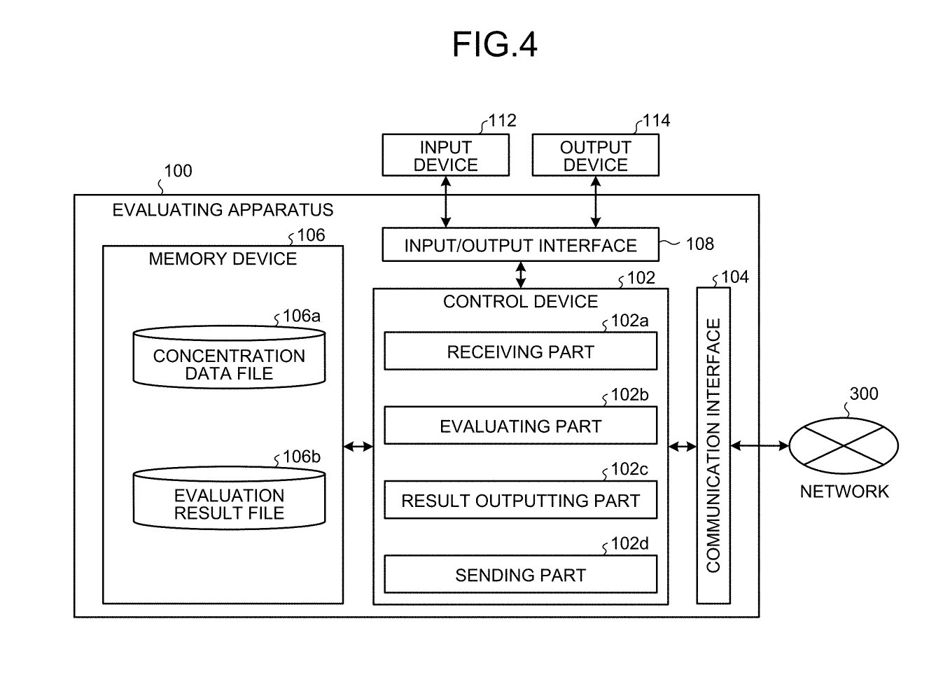

Now, the configuration of the evaluating apparatus 100 in the present system will be described with reference to FIGS. 4 to 7. FIG. 4 is a block diagram showing an example of the configuration of the evaluating apparatus 100 in the present system, showing conceptually only the region relevant to the present invention.

The evaluating apparatus 100 includes (i) a control device 102, such as CPU (Central Processing Unit), that integrally controls the evaluating apparatus, (ii) a communication interface 104 that connects the evaluating apparatus to the network 300 communicatively via communication apparatuses such as a router and wired or wireless communication lines such as a private line, (iii) a memory device 106 that stores various databases, tables, files and others, and (iv) an input/output interface 108 connected to an input device 112 and an output device 114, and these parts are connected to each other communicatively via any communication channel. The evaluating apparatus 100 may be present together with various analyzers (e.g., an amino acid analyzer) in a same housing. For example, the evaluating apparatus 100 may be a compact analyzing device including components (hardware and software) that calculate (measure) the concentration value of at least one of the 23 kinds of amino acids and the 7 kinds of amino acid related metabolites in blood and output (e.g., print or display on a monitor) the calculated concentration value, wherein the compact analyzing device is characterized by further including the evaluating part 102b described later, and using the components to output results obtained by the evaluating part 102b.

The communication interface 104 allows communication between the evaluating apparatus 100 and the network 300 (or a communication apparatus such as a router). Thus, the communication interface 104 has a function to communicate data via a communication line with other terminals.

The input/output interface 108 is connected to the input device 112 and the output device 114. A monitor (including a home television), a speaker, or a printer may be used as the output device 114 (hereinafter, the output device 114 may be described as the monitor 114). A keyboard, a mouse, a microphone, or a monitor functioning as a pointing device together with a mouse may be used as the input device 112.

The memory device 106 is a storage means, and examples thereof include a memory apparatus such as RAM (Random Access Memory) and ROM (Read Only Memory), a fixed disk drive such as a hard disk, a flexible disk, and an optical disk. The memory device 106 stores computer programs giving instructions to the CPU for various processings, together with OS (Operating System). As shown in the figure, the memory device 106 stores the concentration data file 106a and the evaluation result file 106b.

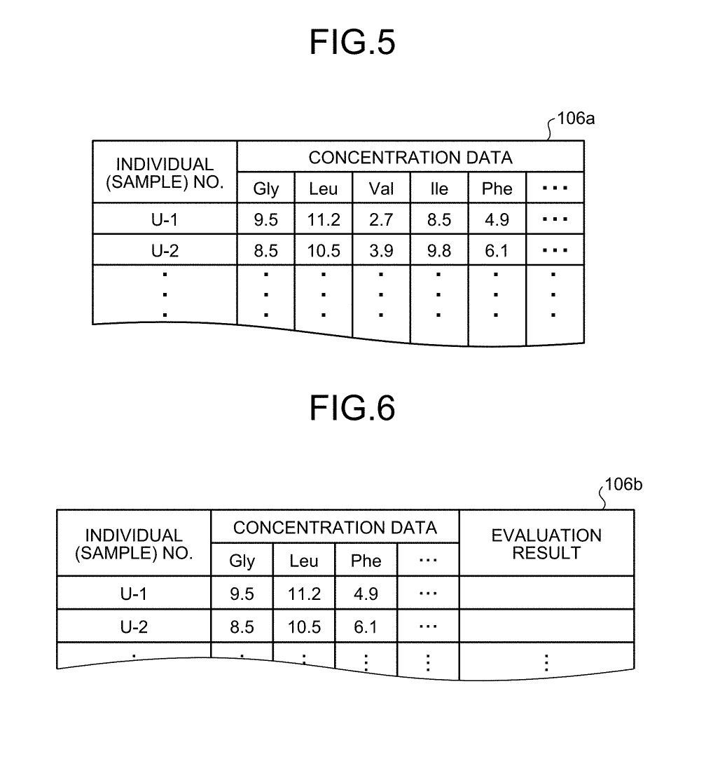

The concentration data file 106a stores the concentration value of at least one of the 23 kinds of amino acids and the 7 kinds of amino acid related metabolites in blood. FIG. 5 is a chart showing an example of information stored in the concentration data file 106a. As shown in FIG. 5, the information stored in the concentration data file 106a includes an individual number for uniquely identifying the individual (sample) as the subject and the concentration data that are correlated to one another. In FIG. 5, the concentration data is assumed to be numerical values, i.e., on a continuous scale, but the concentration data may be expressed on a nominal scale or an ordinal scale. In the case of the nominal or ordinal scale, any number may be allocated to each state for analysis. The concentration data may be combined with the value related to the other biological information.

Returning to FIG. 4, the evaluation result file 106b stores the evaluation results obtained in the evaluating part 102b described below. FIG. 6 is a chart showing an example of information stored in the evaluation result file 106b. The information stored in the evaluation result file 106b includes the individual number for uniquely identifying the individual (sample) as the subject, the previously obtained concentration data of the individual, and the evaluation result on the future risk of developing AD (for example, the converted value of the concentration value by a converting part 102b1 described below, the positional information generated by a generating part 102b2 described below, or the classification result obtained by a classifying part 102b3 described below), that are correlated to one another.

Returning to FIG. 4, the control device 102 has an internal memory storing, for example, control programs such as OS (Operating System), programs for various processing procedures, and other needed data, and performs various information processings according to these programs. As shown in the figure, the control device 102 includes mainly a receiving part 102a, the evaluating part 102b, a result outputting part 102c, and a sending part 102d. The control device 102 performs data processings such as removal of data including defective, removal of data including many outliers, and removal of explanatory variables for the defective-including data in the concentration data transmitted from the client apparatus 200.

The receiving part 102a receives, via the network 300, information (specifically, the concentration data, etc.) transmitted from the client apparatus 200.

The evaluating part 102b evaluates the future risk of developing AD for the individual using the concentration value of at least one of the 23 kinds of amino acids and the 7 kinds of amino acid related metabolites included in the concentration data of the individual received by the receiving part 102a.

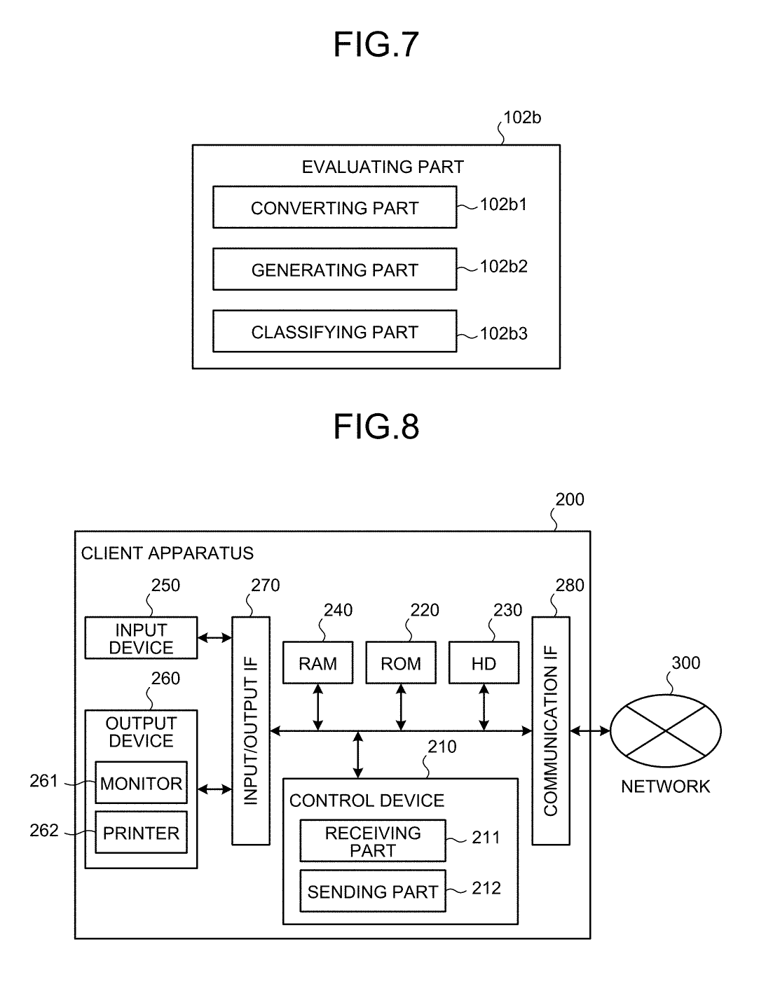

Hereinafter, a configuration of the evaluating part 102b will be described with reference to FIG. 7. FIG. 7 is a block diagram showing the configuration of the evaluating part 102b, and only a part in the configuration related to the present invention is shown conceptually. The evaluating part 102b includes the converting part 102b1, the generating part 102b2, and the classifying part 102b3, additionally.

The converting part 102b1 converts the concentration value of at least one of the 23 kinds of amino acids and the 7 kinds of amino acid related metabolites included in the concentration data, for example, by the conversion method described above. The evaluating part 102b may store the converted value by the converting part 102b1 as the evaluation result in a predetermined region of the evaluation result file 106b.

The generating part 102b2 generates the positional information about the position of the predetermined mark on the predetermined scale visually presented on the display device such as a monitor or the physical medium such as paper, using the concentration value or the converted value of the concentration value by the converting part 102b1. The evaluating part 102b may store the positional information generated by the generating part 102b2 as the evaluation result in a predetermined region of the evaluation result file 106b.

The classifying part 102b3 classifies the individual into any one of the categories defined at least considering the degree of the future risk of developing AD, using the concentration value or the converted value of the concentration value by the converting part 102b1.

The result outputting part 102c outputs, into the output device 114, for example, the processing results in each processing part in the control device 102 (including the evaluation results obtained by the evaluating part 102b).

The sending part 102d is a means for performing data transmission to an external apparatus and, for example, transmits the evaluation results obtained by the evaluating part 102b to the client apparatus 200 that is a sender of the concentration data of the individual.

Hereinafter, a configuration of the client apparatus 200 in the present system will be described with reference to FIG. 8. FIG. 8 is a block diagram showing an example of the configuration of the client apparatus 200 in the present system, and only the part in the configuration relevant to the present invention is shown conceptually.

The client apparatus 200 includes a control device 210, ROM 220, HD (Hard Disk) 230, RAM 240, an input device 250, an output device 260, an input/output IF 270, and a communication IF 280 that are connected communicatively to one another through a communication channel.

The control device 210 has a receiving part 211 and a sending part 212. The receiving part 211 receives various kinds of information such as the evaluation results transmitted from the evaluating apparatus 100, via the communication IF 280. The sending part 212 sends various kinds of information such as the concentration data of the individual, via the communication IF 280, to the evaluating apparatus 100. The control device 210 may include an evaluating part 210a (including a converting part 210a1, a generating part 210a2, and a classifying part 210a3) having the same functions as the functions of the evaluating part 102b in the control device 102 of the evaluating apparatus 100.

The input device 250 is, for example, a keyboard, a mouse, or a microphone. The monitor 261 described below also functions as a pointing device together with a mouse. The output device 260 is an output means for outputting information received via the communication IF 280, and includes the monitor 261 (including home television) and a printer 262. In addition, the output device 260 may have a speaker or the like additionally. The input/output IF 270 is connected to the input device 250 and the output device 260.

The communication IF 280 connects the client apparatus 200 to the network 300 (or communication apparatus such as a router) communicatively. In other words, the client apparatus 200 is connected to the network 300 via a communication apparatus such as a modem, TA (Terminal Adapter) or a router, and a telephone line, or via a private line. In this way, the client apparatus 200 can access to the evaluating apparatus 100 by using a particular protocol.

The client apparatus 200 may be realized by installing softwares (including programs, data and others) for providing various kinds of processing functions included in the control device 210 to an information processing apparatus (for example, an information processing terminal such as a known personal computer, a workstation, a family computer, Internet TV (Television), PHS (Personal Handyphone System) terminal, a mobile phone terminal, a mobile unit communication terminal, or PDA (Personal Digital Assistants)) connected as needed with peripheral devices such as a printer, a monitor, and an image scanner.

All or a part of processings of the control device 210 in the client apparatus 200 may be performed by CPU and programs read and executed by the CPU. Computer programs for giving instructions to the CPU and executing various processings together with the OS (Operating System) are recorded in the ROM 220 or HD 230. The computer programs, which are executed as they are loaded in the RAM 240, constitute the control device 210 with the CPU. The computer programs may be stored in application program servers connected via any network to the client apparatus 200, and the client apparatus 200 may download all or a part of them as needed. All or any part of processings of the control device 210 may be realized by hardware such as wired-logic.

In the above-described present description of the configuration of the evaluating system, the evaluating apparatus 100 executes the reception of the concentration data, the evaluation for the individual based on the concentration data (including the conversion of the concentration value, the generation of the positional information, and the classification of the individual into the category), and the transmission of the evaluation results, while the client apparatus 200 executes the reception of the evaluation results, described as an example. However, when the client apparatus 200 includes the evaluating unit 210a, for example, the conversion of the concentration value, the generation of the positional information, and the classification of the individual into the category may be appropriately shared between the evaluating apparatus 100 and the client apparatus 200. For example, when the client apparatus 200 receives the converted value of the concentration value from the evaluating apparatus 100, the evaluating unit 210a may generate the positional information corresponding to the converted value in the generating unit 210a2 and classify the individual into any one of the categories using the converted value in the classifying unit 210a3. When the client apparatus 200 receives the converted value of the concentration value and the positional information from the evaluating apparatus 100, the evaluating unit 210a may classify the individual into any one of the categories using the converted value in the classifying unit 210a3.

2-3. Other embodiments

In addition to the second embodiment described above, the evaluating apparatus, the evaluating method, the evaluating program product, the evaluating system, and the terminal apparatus according to the present invention can be practiced in various different embodiments within the technological scope of the claims.

Of the processings described in the second embodiment, all or a part of the processings described as automatically performed ones may be manually performed, or all or a part of the processings described as manually performed ones may be also automatically performed by known methods.

In addition, the processing procedures, the control procedures, the specific names, the information including parameters such as registered data of various processings and retrieval conditions, the screen examples, and the database configuration shown in the description and the drawings may be arbitrarily modified unless otherwise specified.

The components of the apparatuses constituting the evaluating system shown in the figures are functionally conceptual and therefore not be physically configured as shown in the figures.

For example, for the operational functions provided in the evaluating apparatus 100, in particular, for the operational functions performed in the control device 102, all or part thereof may be implemented by the CPU (Central Processing Unit) and programs interpreted and executed in the CPU, or may be implemented by wired-logic hardware. The program is recorded in a non-transitory tangible computer-readable recording medium including programmed instructions for making an information processing apparatus execute the evaluating method according to the present invention, and is mechanically read as needed by the evaluating apparatus 100. More specifically, computer programs to give instructions to the CPU in cooperation with the OS (operating system) to perform various processes are recorded in the memory device 106 such as ROM or a HDD (hard disk drive). The computer programs are executed by being loaded to RAM, and form the control unit in cooperation with the CPU.

The computer programs may be stored in an application program server connected to the evaluating apparatus 100 via an arbitrary network, and all or part thereof can be downloaded as necessary.

The evaluating program according to the present invention may be stored in the non-transitory tangible computer-readable recording medium, or can be configured as a program product. The “recording medium” mentioned here includes any “portable physical medium” such as a memory card, a USB (universal serial bus) memory, an SD (secure digital) card, a flexible disk, a magneto-optical disc, ROM, EPROM (erasable programmable read only memory), EEPROM (registered trademark) (electronically erasable and programmable read only memory), CD-ROM (compact disk read only memory), MO (magneto-optical disk), DVD (digital versatile disk), and Blu-ray (registered trademark) Disc.

The “program” mentioned here is a data processing method described in an arbitrary language or description method, and therefore any form such as a source code and a binary code is acceptable. The “program” is not necessarily limited to a program configured as a single unit, and, therefore, includes those dispersively configured as a plurality of modules and libraries and those in which the function of the program is achieved in cooperation with separate programs represented as OS (operating system). Any known configuration and procedures can be used as a specific configuration and reading procedure to read a recording medium by each apparatus shown in the embodiments, an installation procedure after the reading, and the like.

The various databases and the like stored in the memory device is a storage unit such as a memory device such as RAM and ROM, a fixed disk drive such as a hard disk, a flexible disk, or an optical disc. The memory device stores therein various programs, tables, databases, files for Web (World Wide Web) pages, and the like used to perform various processes and to provide Web sites.

The evaluating apparatus 100 may be configured as an information processing apparatus such as known personal computer and work station, or may be configured as the information processing apparatus connected to an arbitrary peripheral device. The evaluating apparatus 100 may be provided by installing software (including the programs and the data, etc.) to cause the information processing apparatus to implement the evaluating method according to the present invention.

Furthermore, a specific configuration of dispersion or integration of the apparatuses is not limited to the shown one. The apparatuses can be configured by functionally or physically dispersing or integrating all or part of the apparatuses in arbitrary units according to various types of additions or the like or according to functional loads. In other words, the embodiments may be implemented in arbitrary combinations thereof or an embodiment may be selectively implemented.

### Example 1

The blood samples of aged persons diagnosed as MCI and the information on dementia diagnosis made after three to five years from the sampling were obtained (from a total of 30 people). Two persons who developed dementia different from AD were excluded and the rest of 28 persons were determined as the subjects. In accordance with the dementia diagnosis information, the 28 persons were classified into an AD group and a non-AD group. The blood samples were measured by the measuring method (A) to determine the blood concentrations (mol/ml) of the 23 kinds of amino acids (α-ABA, Ala, Arg, Asn, Cit, Glu, Gln, Gly, His, Ile, Leu, Lys, Met, Orn, Phe, Pro, Ser, Thr, Trp, Tyr, Val, Cysteine, Taurine). Furthermore, the same blood samples were measured by the above-mentioned measuring method (A) to determine the blood concentrations (mol/ml) of the two kinds of amino acid related metabolites (L-3-Aminoisobutyric acid, N8-Acetylspermidine). The method proposed in 1995 by Petersen et al., from the Mayo clinic was used as the MCI diagnostic criteria (“Petersen, et al., Arch Neurol (1999) 56(6):760.; Mild cognitive impairment: clinical characterization and outcome.”).

For the blood concentrations of substances in the non-AD group and the AD group, in the test with the null hypothesis (Mann-Whitney U test) that “both groups have the same average value”, substances having a significant variation (p-value<0.05) were Ala, α-ABA, L-3-Aminoisobutyric acid, and N8-Acetylspermidine for the non-AD group. Table 1 presents ROC_AUC values of the ROC curve used for distinguishing between the persons without AD and the persons with AD, using the blood concentrations of the above-mentioned four substances three to five years after the sampling mentioned above. Example 1 proved that the above-mentioned four substances are valuable for “evaluating the future risk of developing AD (for example, two-group discrimination determining whether the risk of developing AD in the future (for example, three to five years after blood collection) is high)” for subjects diagnosed as MCI. ROC_AUC is defined as the area under the curve (AUC) in the receiver operating characteristic curve (ROC) created by plotting (x, y)=(1-specificity, sensitivity) on two-dimensional coordinates. The value of ROC_AUC is 1 in perfect discrimination, and the closer this value is to 1, the higher the discriminative characteristic.

TABLE 1

| SUBSTANCE NAME | ROC AUC |
|---|---|
| Ala | 0.7500 |
| α-ABA | 0.6944 |
| L-3-Aminoisobutyric acid | 0.7278 |
| N8-Acetylspermidine | 0.8167 |

### Example 2

Of the aged persons diagnosed as MCI described in Example 1, the females were determined as the subjects (a total of 17 persons). In accordance with the dementia diagnosis information, the 17 persons were classified into an AD group and a non-AD group. The blood samples were measured by the same measuring method as Example 1 to determine the peak areas and the blood concentrations (mol/ml) of the 23 kinds of amino acids and two kinds of amino acid related metabolites (Ethylglycine and 5-Hydroxytryptophan).

For the peak areas and the blood concentrations of substances in the non-AD group and the AD group, in the test with the null hypothesis (Mann-Whitney U test) that “both groups have the same average value”, substances having a significant variation (p-value<0.05) were Ethylglycine and 5-Hydroxytryptophan for the non-AD group. Table 2 presents ROC_AUC values of the ROC curve used for distinguishing between the persons without AD and the persons with AD based on the peak areas and the blood concentrations of the above-mentioned two substances three to five years after the sampling mentioned above. Example 2 proved that the above-mentioned two substances are valuable for “evaluating the future risk of developing AD (for example, two-group discrimination determining whether the risk of developing AD in the future (for example, three to five years after blood collection) is high)” for subjects diagnosed as MCI.

TABLE 2

| SUBSTANCE NAME | ROC AUC |
|---|---|
| Ethylglycine | 0.8333 |
| 5-Hydroxytryptophan | 0.8030 |

### Example 3

Of the aged persons diagnosed as MCI described in Example 1, the males were determined as the subjects (a total of 11 persons). In accordance with the dementia diagnosis information, the 11 persons were classified into an AD group and a non-AD group. The blood samples were measured by the same measuring method as Example 1 to determine the blood concentrations (mol/ml) of the 23 kinds of amino acids and two kinds of amino acid related metabolites (L-3-Aminoisobutyric acid, N(tau)-Methyl-L-histidine).

For the blood concentrations of substances in the non-AD group and the AD group, in the test with the null hypothesis (Mann-Whitney U test) that “both groups have the same average value”, substances having a significant variation (p-value<0.05) were Pro, α-ABA, L-3-Aminoisobutyric acid and N(tau)-Methyl-L-histidine for the non-AD group. Table 3 presents ROC_AUC values of the ROC curve used for distinguishing between the persons without AD and the persons with AD, using the blood concentrations of the above-mentioned four substances three to five years after the sampling mentioned above. Example 3 proved that the above-mentioned four substances are valuable for “evaluating the future risk of developing AD (for example, two-group discrimination determining whether the risk of developing AD in the future (for example, three to five years after blood collection) is high)” for subjects diagnosed as MCI.

TABLE 3

| SUBSTANCE NAME | ROC AUC |
|---|---|
| Pro | 0.7857 |
| α-ABA | 0.9286 |
| L-3-Aminoisobutyric acid | 1.000 |
| N(tau)-Methyl-L-histidine | 0.9643 |

### Example 4

Of the aged persons diagnosed as MCI described in Example 1, the persons without APOEε4 allele that is one of the known risk factors of developing AD were determined as the subjects (a total of 10 persons). In accordance with the dementia diagnosis information, the 10 persons were classified into an AD group and a non-AD group. The blood samples were measured by the same measuring method as Example 1 to determine the blood concentrations (mol/ml) of the 23 kinds of amino acids and Hypotaurine.

For the blood concentrations of substances in the non-AD group and the AD group, in the test with the null hypothesis (Mann-Whitney U test) that “both groups have the same average value”, substances having a significant variation (p-value<0.05) were Val, Leu, Ile, and Hypotaurine for the non-AD group. Furthermore, it was found that the total value of blood concentrations of branched-chain amino acids (Val, Leu, Ile) and the total value of blood concentrations of essential amino acids (His, Ile, Leu, Lys, Met, Val, Phe, Thr, Trp) have significant variations (p-value<0.05). Table 4 presents ROC AUC values of the ROC curve used for distinguishing between the persons without AD and the persons with AD, using the blood concentrations of the above-mentioned four substances and two amino acid groups three to five years after the sampling mentioned above. Example 4 proved that the above-mentioned four substances and two amino acid groups are valuable for “evaluating the future risk of developing AD (for example, two-group discrimination determining whether the risk of developing AD in the future (for example, three to five years after blood collection) is high)” for subjects diagnosed as MCI.

TABLE 4

| SUBSTANCE NAME | ROC AUC |
|---|---|
| Val | 0.8333 |
| Leu | 0.8750 |
| Ile | 0.8333 |
| Hypotaurine | 0.9167 |
| Branched-chain amino acids | 0.8750 |
| (Val + Leu + Ile) | |
| Essential amino acids | 0.8750 |
| (His + Ile + Leu + Lys + Met + Val + | |
| Phe + Thr + Trp) | |

Although the invention has been described with respect to specific embodiments for a complete and clear disclosure, the appended claims are not to be thus limited but are to be construed as embodying all modifications and alternative constructions that may occur to one skilled in the art that fairly fall within the basic teaching herein set forth.

What is claimed is:

1. An evaluating method comprising:
an evaluating step of evaluating future risk of developing Alzheimer's disease for a subject to be evaluated having mild cognitive impairment using a concentration value of at least one of α-ABA, Ala, Arg, Asn, Cit, Gln, Glu, Gly, His, Ile, Leu, Lys, Met, Orn, Phe, Pro, Ser, Thr, Trp, Tyr, Val, Cysteine, Taurine, bABA, Ethylglycine, Hypotaurine, 3-Me-His, 5-HydroxyTrp, aAiBA, and N8-Acetylspermidine in blood of the subject.

2. An evaluating apparatus comprising a control unit, wherein the control unit includes:
an evaluating unit that evaluates future risk of developing Alzheimer's disease for a subject to be evaluated having mild cognitive impairment using a concentration value of at least one of α-ABA, Ala, Arg, Asn, Cit, Gln, Glu, Gly, His, Ile, Leu, Lys, Met, Orn, Phe, Pro, Ser, Thr, Trp, Tyr, Val, Cysteine, Taurine, bABA, Ethylglycine, Hypotaurine, 3-Me-His, 5-HydroxyTrp, aAiBA, and N8-Acetylspermidine in blood of the subject.

3. An evaluating method executed by an information processing apparatus including a control unit, wherein
the evaluating method comprises an evaluating step of evaluating future risk of developing Alzheimer's disease for a subject to be evaluated having mild cognitive impairment using a concentration value of at least one of α-ABA, Ala, Arg, Asn, Cit, Gln, Glu, Gly, His, Ile, Leu, Lys, Met, Orn, Phe, Pro, Ser, Thr, Trp, Tyr, Val, Cysteine, Taurine, bABA, Ethylglycine, Hypotaurine, 3-Me-His, 5-HydroxyTrp, aAiBA, and N8-Acetylspermidine in blood of the subject, wherein the evaluating step is executed by the control unit.

4. An evaluating program product having a non-transitory tangible computer readable medium including a programmed instruction for making an information processing apparatus including a control unit execute an evaluating method, wherein the evaluating method comprises an evaluating step of evaluating future risk of developing Alzheimer's disease for a subject to be evaluated having mild cognitive impairment using a concentration value of at least one of α-ABA, Ala, Arg, Asn, Cit, Gln, Glu, Gly, His, Ile, Leu, Lys, Met, Orn, Phe, Pro, Ser, Thr, Trp, Tyr, Val, Cysteine, Taurine, bABA, Ethylglycine, Hypotaurine, 3-Me-His, 5-HydroxyTrp, aAiBA, and N8-Acetylspermidine in blood of the subject.

5. An evaluating system comprising an evaluating apparatus including a control unit and a terminal apparatus including a control unit that are connected to each other communicatively via a network, wherein
the control unit of the terminal apparatus includes:
a concentration data-sending unit that transmits concentration data on a concentration value of at least one of α-ABA, Ala, Arg, Asn, Cit, Gln, Glu, Gly, His, Ile, Leu, Lys, Met, Orn, Phe, Pro, Ser, Thr, Trp, Tyr, Val, Cysteine, Taurine, bABA, Ethylglycine, Hypotaurine, 3-Me-His, 5-HydroxyTrp, aAiBA, and N8-Acetylspermidine in blood of a subject to be evaluated having mild cognitive impairment, to the evaluating apparatus; and
a result-receiving unit that receives an evaluation result on future risk of developing Alzheimer's disease for the subject, transmitted from the evaluating apparatus, and
the control unit of the evaluating apparatus includes:
a concentration data-receiving unit that receives the concentration data transmitted from the terminal apparatus;
an evaluating unit that evaluates the future risk of developing Alzheimer's disease for the subject using the concentration value of the at least one of the amino acids and the amino acid related metabolites included in the concentration data received by the concentration data-receiving unit; and
a result-sending unit that transmits the evaluation result obtained by the evaluating unit to the terminal apparatus.

6. A terminal apparatus comprising a control unit, wherein the control unit includes a result-obtaining unit that obtains an evaluation result on future risk of developing Alzheimer's disease for a subject to be evaluated having mild cognitive impairment,
wherein the evaluation result is the result of evaluating the future risk of developing Alzheimer's disease for the subject using a concentration value of at least one of α-ABA, Ala, Arg, Asn, Cit, Gln, Glu, Gly, His, Ile, Leu, Lys, Met, Orn, Phe, Pro, Ser, Thr, Trp, Tyr, Val, Cysteine, Taurine, bABA, Ethylglycine, Hypotaurine, 3-Me-His, 5-HydroxyTrp, aAiBA, and N8-Acetylspermidine in blood of the subject.

7. An evaluating apparatus comprising a control unit, being connected to a terminal apparatus communicatively via a network, wherein the control unit includes:
a concentration data-receiving unit that receives concentration data on a concentration value of at least one of α-ABA, Ala, Arg, Asn, Cit, Gln, Glu, Gly, His, Ile, Leu, Lys, Met, Orn, Phe, Pro, Ser, Thr, Trp, Tyr, Val, Cysteine, Taurine, bABA, Ethylglycine, Hypotaurine, 3-Me-His, 5-HydroxyTrp, aAiBA, and N8-Acetylspermidine in blood of a subject to be evaluated having mild cognitive impairment, transmitted from the terminal apparatus;
an evaluating unit that evaluates future risk of developing Alzheimer's disease for the subject using the concentration value of the at least one of the amino acids and the amino acid related metabolites included in the concentration data received by the concentration data-receiving unit; and
a result-sending unit that transmits an evaluation result obtained by the evaluating unit to the terminal apparatus.
